(19) United States
(12) Patent Application Publication
(10) Pub. No.: US 2007/0141060 A1
(43) Pub. Date: Jun. 21, 2007

(54) Hmgb1 protein inhibitors for the regulation of smooth muscle cells and endothelial cells profileration

(75) Inventor: Marco E. Bianchi, Peschiera Borromeo (IT)

Correspondence Address: YOUNG & THOMPSON, 745 SOUTH 23RD STREET, 2ND FLOOR, ARLINGTON, VA, 22202, US

(73) Assignees: MARCO E BIANCHI, Peschiera Borromeo (IT); BIO3 RESEARCH SRL, Milano (IT)

(21) Appl. No.: 10/586,752

(22) PCT Filed: Jan. 26, 2005
(86) PCT No.: PCT/IT05/00039

§ 371 Date: Aug. 17, 2006

(30) Foreign Application Priority Data
Feb. 3, 2004 (IT) RM2004A000058

(51) Int. Cl. A61K 39/395

(52) U.S. Cl. 424/146.1; 514/12

(57) ABSTRACT

The use of molecules that modulate the action of HMGB1 protein (or molecules having sequence homology) for the preparation of therapeutic agents for the treatment of diseases to related to endothelial and smooth muscle cells proliferation; said proliferation can be due to endothelium damage, caused by surgical operation and manipulation made on or through blood vessels, including coronary and/or carotid angioplasty, angiographic surgery, and surgery using catheters.

The present invention refers to the field of molecular biology and in particular to the use of HMGB1 protein inhibitors and antagonists for the preparation of therapeutic agents for prevention and treatment of diseases related to proliferation of endothelial and smooth muscle cells, especially vascular diseases, including those events that occur after coronary and/or carotid angioplasty, with or without stent positioning, angiographic surgery, and surgery using catheters.

As previously described in PCT application No. PCT/IT02/00153, HMGB1 has been identified as a chemoattractants for smooth muscle cells and fibroblasts.

Tissue remodelling of concentric tissue layers of blood vessels, and particularly of smooth muscle cells, is the key mechanism of blood vessels restriction, that lead to reduction of blood flux and plaque and thrombos formation, for this reason HMGB1 protein antagonists and inhibitors can be advantageously used for the treatment of vascular diseases including those events that occur after coronary and/or carotid angioplasty, with or without stent positioning and angiographic surgery.

HMGB1 is released after every mechanical injury that induce cellular necrosis, consequently all surgical operation made on or through blood vessels damage endothelial cells that coat their internal surface.

The present invention demonstrates that HMGB1 has a strong biological effect of blood vessel cells: HMGB1 induces both smooth muscle cells (SMC) and endothelial cells proliferation.

Thus, HMGB1 protein antagonists and inhibitors can advantageously be used to modulate blood vessel cells proliferation.

Endothelial cells cover the internal lumen of arterial, venous and lymphatic vessels. SMC cells are predominant in blood vessels having large diameter, they resided in tunica media surrounded by extracellular matrix. In intact vessels SMC are in a contracted state, they show a phenotype without cellular divisions and with no migratory activity, providing vessel walls rigidity and elasticity and controlling blood pressure.

When endothelium is damaged by a mechanical injury or a inflammatory response, SMC cells change towards the synthetic phenotype and start cellular division and proliferation.

Our data show that the change toward the synthetic phenotype is induced by HMGB1.

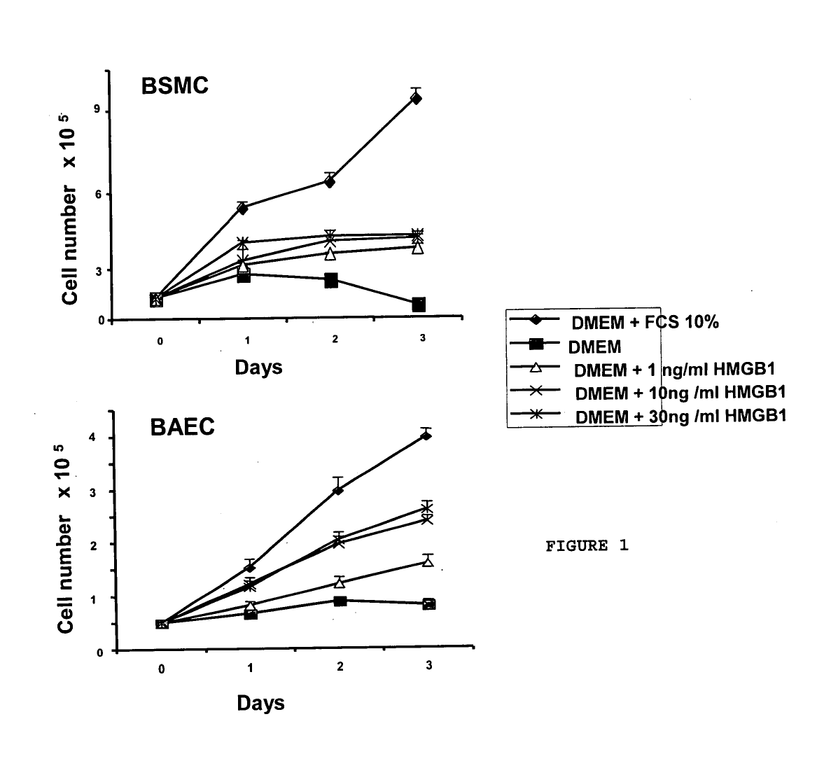

This evidence is underlined by the following experiment and the results summarised in FIG. 1.

HMGB1 Induces Endothelial and Smooth Muscle Cells Proliferation.

Bovine aorta endothelial cells or bovine smooth muscle at an early passage were cultured in DMEM+10% FCS. Cells were then kept for 16 hours in DMEM in absence of FCS, and plated in 6 cm wells of 6-well plates (about 50 000 cells per well). Cells were then cultured, in triplicate, in the presence of DMEM alone, DMEM+10% FCS, or DMEM+different concentrations of HMGB1. At 24 hours intervals cells were counted under the microscope.

FIG. 1 shows the results: cells kept in DMEM without additions grew very little, while cells in the presence of serum kept dividing (about 1 division every 24 hours for BAEC, and every 18 hours for BSMC).

HMGB1 caused BAEC to keep dividing, while it caused a couple of rounds of division in BSMC, followed by quiescence.

Repeated addition of HMGB1 to BSMC, every 24 hours, caused the cells to keep proliferating (data not shown).

Antibodies against HMGB1 (100 μg/ml) abolished the effect of HMGB1 (data not shown).

In similar conditions, HMGB1 does not cause the proliferation of 3T3 mouse fibroblasts (data not shown).

The above experiments show that HMGB1 induces smooth muscle cells and endothelial cells proliferation.

As known, HMGB1 is released after mechanical injury of cells (Degryse et al, 2001; Scaffidi et al., 2002), thus HMGB1 shows all the typical features of a molecule able to facilitate atherosclerosis and/or restenosis due to blood vessels damage.

In view of the above, it is evident that every kind of molecules that modulate or block the interaction between HMGB1 and its RAGE receptor (or receptors if more that one are present) can efficiently be used for the production of pharmacological preparation for the treatment of diseases related to cellular proliferation, in particular of endothelial and smooth muscle cells. For example, those events that occur after coronary and/or carotid angioplasty, angiographic surgery, and surgery using catheters.

RAGE (receptor for advanced glycation endproducts) is a HMGB1 receptor, but others have been identified or suggested (Park et al., 2003).

Object of the present invention are: all kind of molecules able to modulate the interaction between HMGB1 and its receptors including all the molecules belonging to the inhibitors class (antibodies or antibodies fragments, fourway DNA) and all the molecules belonging to the antagonist class (HMGB1 fragments molecules or molecules with similar sequence).

Said molecules, that bind or inhibit HMGB1, can be injected or released by instruments used for coronary and/or carotid angioplastic surgery (catheters, surgery instruments, implants or stents) or said molecules can be bound to the instruments' surface.

### BIBLIOGRAPHY

Bianchi M E, Bonaldi T, Scaffidi P, Müller, S, Degryse B (2002) HMGB1 protein inhibitors and/or antagonists for the treatment of vascular diseases. Publication number WO 02/074337.
Degryse B, Bonaldi T, Scaffidi P, Müller S, Resnati M, Sanvito F, Arrigoni G and Bianchi M E (2001) The High Mobility Group (HMG) boxes of the nuclear protein HMG1 induce chemotaxis and cytoskeleton reorganization in rat smooth muscle cells. J Cell Biol 152: 1197-2006.
Park et al., Involvement of TLR 2 and TLR 4 in cellular activation by high mobility group box 1 protein (HMGB1). J Biol Chem, published online December 2003; 10.1074/jbc.M306793200
Scaffidi P. Misteli T and Bianchi M E (2002) Release of chromatin protein HMGB1 by necrotic cells triggers inflammation. Nature 418: 191-5.

1-11. (canceled)

12. Use of HMG box binding molecules for the preparation of therapeutic agents for the treatment of vascular diseases related to endothelial and smooth muscle cells proliferation.

13. Use of HMG box binding molecules for the preparation of therapeutic agents for the treatment of vascular diseases related to endothelial and smooth muscle cells proliferation according to claim 12 characterised in that vascular diseases are due to atherosclerosis and/or restenosis after blood vessels damage.

14. Use of HMG box binding molecules for the preparation of therapeutic agents for the treatment of vascular diseases related to endothelial and smooth muscle cells proliferation according to claim 12 characterised in that vascular diseases are due to atherosclerosis and/or restenosis after blood vessels damage, including those events that occur after coronary and/or carotid angioplasty, with or without stent positioning, angiographic surgery, and surgery using catheters.

15. Use of HMG box binding molecules according to claim 12 wherein said molecules belong to the group comprising antibodies or antibodies fragments, inhibitors and four-way DNA.

16. Therapeutic agent for the treatment of vascular diseases related to smooth muscle and endothelial cells proliferation characterised in that it comprises a therapeutic active amount of HMG box binding molecules.

17. Therapeutic agent for the treatment of vascular diseases related to smooth muscle and endothelial cells proliferation according to claim 16 wherein said molecules belong to the group comprising antibodies or antibodies fragments, inhibitors and four-way DNA and not sRAGE.

18. Therapeutic agent for the treatment of vascular diseases related to smooth muscle and endothelial cells proliferation characterised in that it comprises a therapeutic active amount of antagonist molecules having sequence homology with HMG box and being able to bind the functional HMG box binding domain of the receptor.

19. Therapeutic agent according to claim 16 characterised in that it is released by catheters, surgical instruments implants or stents.
